SEQUENCE LISTING (2 sequences)

SEQ ID NO: 1 - protein, 105 aa, Homo sapiens
MVKQIESKTAFQEALDAAGDKLVVVDFSATWCGPCKMIKPFFHSLSEKYSNVIFLEVDVDDCQDVASECEVKCMPTFQFFKKGQKVGEFSGANKEKLEATINELV

SEQ ID NO: 2 - DNA, 318 bp, Homo sapiens
atggtgaagcagatcgagagcaagactgcttttcaggaagccttggacgctgcaggtgataaacttgtagtagttgacttctcagccacgtggtgtgggccttgcaaaatgatcaagcctttctttcattccctctctgaaaagtattccaacgtgatattccttgaagtagatgtggatgactgtcaggatgttgcttcagagtgtgaagtcaaatgcatgccaacattccagttttttaagaagggacaaaaggtgggtgaattttctggagccaataaggaaaagcttgaagccaccattaatgaattagtctaa

(19) United States
(12) Patent Application Publication
(10) Pub. No.: US 2007/0264248 A1
(43) Pub. Date: Nov. 15, 2007

(54) Preparation for preventing or treating disorders caused by toxic constituents contained in fiber combustion smoke

(75) Inventors: Junji Yodoi, Kyoto (JP); Michiaki Mishima, Kyoto (JP); Yuma Hoshino, Kyoto (JP); Hajime Nakamura, Osaka (JP); Tomijiro Hara, Kyoto (JP); Makiko Narita, Kyoto (JP)

Correspondence Address: BROWN & MICHAELS, PC;400 M & T BANK BUILDING, 118 NORTH TIOGA ST, ITHACA, NY, 14850, US

(73) Assignee: Redox Bioscience Inc., Kyoto (JP)

(21) Appl. No.: 11/438,863

(22) Filed: May 23, 2006

(30) Foreign Application Priority Data
May 12, 2006 (JP) 2006134507

(51) Int. Cl. A61K 38/44

(52) U.S. Cl. 424/94.4

(57) ABSTRACT

Preventive or therapeutic agents for controlling various disorders induced by toxic substances in smoke produced by the combustion of fibers such as tobacco are disclosed. A polypeptide of the thioredoxin family (TRX) prevents toxic substances in smoke, produced by the combustion of fibers such as tobacco, from inducing inflammatory reactions and tissue fibrosis. Accordingly, TRX is effective for controlling of a wide range of disorders caused by toxic substances contained in smoke produced by combustion of fibers. The object of the present invention is to provide the preventive or therapeutic agents for disorders caused by toxic substances contained in smoke produced by combustion of fibers comprising an effective amount of polypeptide of thioredoxin family and/or one or more of its inducers.

### BACKGROUND OF THE INVENTION

1. Field of the Invention

The present invention relates to agents for preventing or treating disorders caused by toxic substances contained in smoke produced by the combustion of fibers that contain one or more material selected from a group consisting of polypeptide of the thioredoxin family (referred to below as “TRX”) and its inducers as active ingredients.

The present invention also relates to pharmaceutical formulations for preventing or treating these disorders that contain one or more material selected from a group consisting of polypeptide of the thioredoxin family and its inducers.

2. Description of Related Art

Smoke produced by the combustion of fibers like tobacco contains many toxic substances including carbon monoxide, nitrogen monoxide, nicotine, and tar. These toxic substances are known to reduce respiratory and immune functions.

In addition, long-standing inhalation or absorption of such toxic smoke components has been reported to increase the risk for ischemic heart disease (such as angina pectoris, myocardial infarction), pulmonary dysfunction, and cancer.

Moreover, inhalation of the smoke containing toxic substances by pregnant women is known to exert adverse effects not only on themselves but also on their fetuses.

Concerning tobacco smoke, in particular, adverse effects of inhalation of smoke that escapes from the lit end of the cigarette (passive smoking) have been reported. Chronic bronchitis, reduced respiratory function, bronchial asthma, arteriosclerosis, and angina pectoris observed in nonsmoking family members of long-standing smokers are considered to be closely related to passive smoking.

Firefighters and persons who experienced being trapped by a fire are likely to develop respiratory disorders due to the large amount of smoke they have inhaled. Passive smoking and inhalation of smoke at fire sites are of a serious problem because it is difficult to avoid inhaling smoke in these cases.

Tobacco smoke is known to be a major cause of lung diseases such as chronic obstructive pulmonary disease (COPD) and lung cancer.

Recently, however, it has also been reported that tobacco smoke induces inflammatory reactions due to increased oxidative stress and acts as one of the factors for inducing generalized inflammation such as arteriosclerosis, coronary artery disease, and lifestyle-related diseases. Smoking is known to be a factor for disorders of not only specific organs (e.g., lungs) but also the entire body.

Thus there is a need for developing agents that is effective for preventing or treating various disorders induced by toxic substances contained in smoke produced by the combustion of fibers like tobacco.

Thioredoxin is a multi-functional peptide with a molecular weight of 12 kDa having a redox activity derived from the disulfide/dithiol exchange reaction between the two cysteine residues in its active amino acid sequence (-Cys-Gly-Pro-Cys-). Thioredoxin plays as an enzyme an important role in the synthesis of deoxyribonucleotide, which supplies hydrogen ion to ribonucleotide reductase. Thioredoxin was first isolated from Escherichia coli and has been isolated and identified from many prokaryotes and eukaryotes.

Adult T-cell leukemia-derived factor (ADF) is a human thioredoxin first identified by the inventors of the present invention as an IL-2 receptor inducing factor produced by T-lymphocytes infected by HTLV-1.

Intracellular thioredoxin plays an important role in the radical scavenging and control of transcription factors involved in redox reactions such as activator protein-1 (AP-1) and nuclear factor-kappa B (NF-κB).

Human thioredoxin controls signal transmission of p38 mitogen activating protein kinase (MAPK) and of apoptosis signal regulating kinase-1 (ASK-1).

Thioredoxin is released into the extracellular space and shows a cytokine-like or chemokine-like actions and that extracellular TRX moves into cells.

However, no report has been made on the relationship between the disorders induced by toxic substances in smoke produced by the combustion of fibers like tobacco and polypeptide of the thioredoxin family (TRX).

### SUMMARY OF THE INVENTION

It is an object of the present invention to provide preventive or therapeutic agents that are effective against various disorders induced by toxic substances in smoke produced by the combustion of fibers like tobacco smoke and that are relatively free of adverse effects.

Polypeptide of the thioredoxin family (TRX) inhibits inflammatory reaction induced by toxic substance that are produced by the combustion of fibers like tobacco and that TRX inhibits tissue fibrosis caused by such toxic substance. TRX is effective for the prevention and treatment of a wide range of disorders caused by toxic substances in smoke produced by the combustion of fibers and completed the present invention.

The present invention provides a preventive or therapeutic agent for disorders caused by toxic substances contained in smoke produced by combustion of fibers, comprising an effective amount of at least one material selected from a group consisting of polypeptide of the thioredoxin family and inducers thereof, as active ingredients.

In one aspect of the present invention, the polypeptide of the thioredoxin family is human thioredoxin.

In one aspect of the present invention, the fiber is selected from a group consisting of tobacco, wood, and paper.

In one aspect of the present invention, the disorders are inflammation in at least one organ selected from a group consisting of respiratory organs and digestive organs.

In one aspect of the present invention, the respiratory organs are at least one organ selected from a group consisting of nasal cavity, paranasal sinuses, pharynx, larynx, trachea, bronchi, and pulmonary alveoli.

In one aspect of the present invention, the digestive organs are at least one organ selected from a group consisting of esophagus, stomach, duodenum, liver, small intestine, and large intestine.

In one aspect of the present invention, the disorder is chronic obstructive pulmonary disease or bronchial asthma.

The present invention further provides a preventive or therapeutic formulation for disorders caused by toxic substances contained in smoke produced by combustion of fibers, comprising an effective amount of at least one material selected from a group consisting of a polypeptide of the thioredoxin family and inducers thereof, as active ingredients.

In one aspect of the present invention, the inducer is sulforaphane.

In one aspect of the present invention, the preventive or therapeutic formulation is foods or beverages.

Since the preventive or therapeutic agents of the present invention contain polypeptide of the thioredoxin family and/or one or more of its inducers, they can suppress inflammation induced by toxic substances contained in smoke produced by the combustion of fibers like tobacco and prevent tissue fibrosis caused by such toxic substances.

Therefore, they are effective against a wide range of disorders caused by toxic substances contained in smoke produced by the combustion of fibers.

In addition, since the preventive or therapeutic agents of the present invention contain TRX as an active ingredient, which is an endogenous thiol protein expressed in the body, or substances that induce the expression of TRX in the body, they are expected to be very safe preventive or therapeutic agents with little possibility of adverse effects.

### BRIEF DESCRIPTION OF THE DRAWING

FIG. 1 shows micrographs of lung sections taken from TRX-Tg mice and from C57BL/6 mice after exposure to tobacco smoke for three days.

FIG. 2 shows neutrophil (PMN) count and total cell count in BALF taken from smoke-exposed TRX-Tg mice and from smoke-exposed C57BL/6 mice.

FIG. 3 shows neutrophil (PMN) counts and total cell counts in BALF taken from C57BL/6 mice administered TRX and in BALF taken from C57BL/6 mice administered physiologic saline.

FIG. 4a shows mRNA transcription activities and suppressive effects on mRNA transcription of MIF in spleen tissues taken from smoke-exposed TRX-Tg mice and from smoke-exposed C57BL/6 mice.

FIG. 4b shows mRNA transcription activities and suppressive effects on mRNA transcription of TNF-α in spleen tissues taken from smoke-exposed TRX-Tg mice and from smoke-exposed C57BL/6 mice.

FIG. 5a shows the expression of MIF protein and TNF-α protein in spleen tissues of smoke-exposed TRX-Tg mice and of smoke-exposed C57BL/6 mice evaluated by Western blotting.

FIG. 5b shows the expression of MIF protein and TNF-α protein in spleen tissues of smoke-exposed TRX-Tg mice and of smoke-exposed C57BL/6 mice evaluated by a densitometric method.

FIG. 6a shows oxidative stress in the heart of smoke-exposed TRX-Tg mice and of smoke-exposed C57BL/6 mice.

FIG. 6b shows oxidative stress in the liver of smoke-exposed TRX-Tg mice and of smoke-exposed C57BL/6 mice.

FIG. 7 shows the expression of MMP-12 mRNA in lung tissues of smoke-exposed TRX-Tg mice and of smoke-exposed C57BL/6 mice.

FIG. 8 shows micrographs of lung sections of TRX-Tg mice and of C57BL/6 mice after long-term (six months) exposure to tobacco smoke.

### DETAILED DESCRIPTION OF THE INVENTION

New functions of polypeptide of the thioredoxin family (TRX) are as follows. TRX prevents toxic substances contained in smoke produced by the combustion of fibers like tobacco from inducing (1) the neutrophilia and (2) the expression of macrophage migration inhibitory factor (MIF) and of tumor necrosis factor-alpha (TNF-α), which are both early inflammatory cytokines. Thus, TRX was considered to reduce inflammatory reactions caused by such toxic substances.

TRX also prevents toxic substances contained in smoke produced by the combustion of fibers from inducing (3) the expression of matrix metalloproteinase-12 (MMP-12). Because it was found TRX could suppress destructions of an alveolar construction in an early stage of inflammation which is caused by MMP-12, it was concluded that TRX could reduce tissue fibrosis in various organs.

Therefore, TRX is effective for preventing and treating a wide range of disorders caused by toxic substances contained in smoke produced by the combustion of fibers (e.g., disorders induced by smoking) because TRX could prevent toxic substances contained in smoke produced by the combustions of fibers from inducing inflammatory reactions and tissue fibrosis. The preferred embodiments of the preventive or therapeutic agents comprising as active ingredients an effective amount of polypeptide of the thioredoxin family and/or its inducers according to present invention will be described in detail below.

TRX

In addition to the human thioredoxin, thioredoxin in the present invention may be any compound belonging to the “thioredoxin family”. Such compounds can be, for example, those having at their active center polypeptide with the following amino acid sequences: -Cys-Gly-Pro-Cys-, -Cys-Pro-Tyr-Cys-, -Cys-Pro-His-Cys-, and -Cys-Pro-Pro-Cys-.

Among these compounds, thioredoxin having the sequence -Cys-Gly-Pro-Cys- at its active center or thioredoxin 2 (mitochondria-specific thioredoxin) is preferable.

Thioredoxin to be used in the present invention may be of any origin, as long as it has such active center as described above. For example, it may be thioredoxin of animals including humans (ADF of animals including humans), bacterial thioredoxin such as that of Escherichia coli, and the like, yeast thioredoxin; polypeptide having a human ADF activity (human ADFP); glutaredoxin and the like of humans or Escherichia coli, and the like. Among these, human thioredoxin and yeast thioredoxin are preferable. Yeast thioredoxin may be isolated from yeast, but yeast containing a high level of thioredoxin may also be used as it is.

Human thioredoxin (hTRX), as used herein, includes polypeptide consisting of 105 amino acids shown as Sequence No. 1. The base sequence of hTRX is shown as Sequence No. 2.

The human thioredoxin in the present invention may also be a modified TRX prepared by a known technique of genetic engineering from hTRX with Sequence No. 1 as long as it suppresses disorders due to toxic substances contained in smoke produced by the combustion of fibers.

Such modified TRX may have a sequence in which one or more amino acids, preferably one or several amino acids, are replaced, lost, added, or inserted in the positions other than either position 32 or position 35 of Sequence No. 1, preferably in the positions other than positions 32-35 of Sequence No. 1.

In the present invention, the above-described one or more types of polypeptide (TRX) of thioredoxin family may be used individually or in combinations.

Fibers, as used herein, include the fibrous parts of plants such as the leaves, trunk, stalks, and roots. Smoke produced by the combustion of fibers includes smoke that is produced when materials such as tobacco, wood, and paper are burned.

Tobacco includes cigarettes, cigars, and chewing tobacco. Wood includes materials compounded with, for example, plywood and resin, as well as naturally occurring wood. Paper includes paper that is compounded with resin and the like.

Toxic substances contained in smoke produced by the combustion of fibers include, for example, nicotine, tar, benzpyrene, toluene, phenol, methylnaphthalene, pyrene, aniline, 2-naphthylamine, carbon dioxide, carbon monoxide, methane, acetylene, ammonia, acetaldehyde, hydrogen cyamide, methylfuran, acetonitrile, pyridine, and dimethylnitrosamine.

Disorders caused by toxic substances contained in smoke produced by the combustion of fibers include disorders induced by inhalation or absorption of such toxic substances. Inhalation of toxic substances, as used herein, includes both active inhalation (e.g., inhalation of tobacco smoke through a cigarette) and passive inhalation (e.g., inhalation of tobacco smoke escaping from the lit end of a cigarette). Inhalation or absorption of such toxic substances may be acute inhalation/absorption (one short inhalation/absorption to several-day-inhalation/absorption), subacute inhalation/absorption (one to three weeks), or chronic inhalation/absorption (several months or longer).

The preventive or therapeutic agents of the present invention may be administered orally or non-orally depending on instructions of clinicians. Thioredoxin, one of the active ingredients of the agents according to the present invention, may also be administered alone or with a common vehicle.

When preventive or therapeutic agents of the present invention are orally administered, it may be administered as solid preparations (e.g., tablets, pills, powders, coated tablets, granules, and capsules), liquid preparations (e.g., solutions, suspensions, emulsions, and syrups), inhalant preparations (e.g., aerosols, atomizers, and nebulizers), or liposome-encapsulated preparations.

For practical use of the preventive or therapeutic agents according to the present invention, they are made into as drugs using pharmaceutically tolerable vehicles. Such pharmaceutical vehicles may be, for example, binders, disintegrators, surfactants, absorption promoters, moisture retainers, adsorbents, lubricants, fillers, volume expanders, moisteners, antiseptics, stabilizers, emulsifiers, solubilizers, salts to control the osmotic pressure, and diluents or fillers such as buffers. These are used selectively according to the dosing units of the agents.

To form the drugs containing the preventive or therapeutic agents of the present invention into tablets, the pharmaceutical vehicles can be, for example, fillers such as lactose, white sugar, sodium chloride, glucose, urea, starch, calcium carbonate, kaolin, crystalline cellulose, silicate, and potassium phosphate; binders such as water, ethanol, propanol, simple syrup, glucose solution, starch suspension, gelatin solution, carboxy methylcellulose, hydroxyl propylcellulose, methylcellulose, and polyvinylpyrrolidone; disintegrators such as carboxy methylcellulose sodium, carboxy methylcellulose calcium, low-grade substituted hydroxy propylcellulose, dry starch, sodium arginate, agar powder, laminaran powder, sodium hydrogen carbonate, and calcium carbonate; surfactants such as polyoxyethylene sorbitan fatty acid esters, sodium lauryl sulfate, and monoglyceride stearate; disintegration inhibitors such as white sugar, stearin, cacao butter, and hydrogen-added oil; absorption promoters such as class 4 ammonium base and sodium lauryl sulfate; moisture retainers such as glycerin and starch; absorbents such as starch, lactose, kaolin, bentonite, and colloid silicate; and lubricants such as purified talc, stearate, boric acid power, and polyethylene glycol.

In addition, such tablets may be, if necessary, coated with a conventional coating. For example, the tablets may be prepared as sugar-coated tablets, gelatin-coated tablets, enteric-coated tablets, or film-coated tablets. The tablets may also be coated with two or more coatings to be double-layered or multi-layered.

To form such drugs into pills, the pharmaceutical vehicles can be, for example, fillers such as glucose, lactose, starch, cacao fat, hardened plant oil, kaolin, and talc; binders such as Arabic gum powder, tragacanth power, gelatin, and ethanol; and disintegrators such as laminaran and agar.

To administer preventive or therapeutic agents of the present invention non-orally, the agents can be used, for example, in the forms of injection preparation (solutions, emulsions, suspensions, etc.) for intravenous, subcutaneous, intradermal, intramuscular, or intraperitoneal injection, solutions (e.g., eye drops, nasal solutions), suspensions, emulsions, instillation agents, and inhalants (aerosol agents, powder inhalants, etc.).

When preventive or therapeutic agents of the present invention are prepared as injections such as solutions, emulsions, and suspensions, they should preferably be sterilized and adjusted to be isotonic to blood. To prepare the therapeutic or preventive agents of the present invention into such dosage forms, diluting agents may be used. The diluting agents can be, for example, water, ethyl alcohol, macrogol, propylene glycol, ethoxy isostearyl alcohol, polyoxy isostearyl alcohol, and polyoxy ethylene sorbitan fatty acid esters.

In this process, salt, glucose, or glycerin may be added to pharmaceutical agents of the present invention in amounts sufficient for adjusting the solution to be isotonic to blood.

Common solubilizers, buffers, soothing agents and the like may also be added to the solution.

When preventive or therapeutic agents of the present invention are provided as liquid agents, they may be preserved after removing water by freezing or by freeze-drying. The freeze-dried agents are redissolved with, for example, distilled water for injection before use.

When preventive or therapeutic agents of the present invention are used as inhalations, they may be prepared using widely used conventional additives for inhalations. Such additives include, for example, propellants; solid fillers such as white sugar, lactose, glucose, mannite, and sorbite; liquid fillers or inactive liquids such as propylene glycol; binders such as methylcellulose, hydroxyl propylcellulose, polyvinylpyrrolidone, polyethylene glycol, and white sugar; lubricants such as magnesium stearate, light silicic anhydride, talc, and sodium rauryl sulfate; preservatives such as sodium benzoate, sodium hydrogen nitrite, methylparaben and propylparaben; stabilizers such as citric acid and sodium citrate; suspending agents such as methylcellulose, polyvinylpyrrolidone, lecithin, and sobitan trioleate; dispersing agents such as surfactants; solvents such as water; tonicity agents such as sodium chloride; pH adjusting agents such as sulfuric acid and hydrochloric acid; and solubilizing agents such as ethanol.

Furthermore, while preparing the preventive or therapeutic agents of the present invention, the agents may be mixed, if necessary, with coloring agents, preservatives, flavoring agents, seasoning agents, sweetening agents, or other drugs.

The effective dose of polypeptide of the TRX family may be determined easily by those skilled in the art based on conventional techniques. For example, the effective dose for an adult is about 0.001-100 mg/kg/day, more preferably about 0.01-10 mg/kg/day, and most preferably about 0.1-10 mg/kg/day.

The daily dose may be administered at one time or in several portions. The dose is preferably adjusted according to the drug form, patient's sexuality and age, and severity of the disorder.

TRX Inducers

As the preventive or therapeutic agents of the present invention, materials that induce TRX in the body can be used for suppressing disorders caused by toxic substances contained in smoke produced by the combustion of fibers. One of the examples of such materials is sulfotaphane.

Sulforafan is contained in, for example, cabbage, purple cabbage, broccoli, kale, rocket, cauliflower, radish, Chinese cabbage, turnip, komatsuna (Brassica campestris var. peruviridis), chingensai (Brassica Chinensis), and the like. Preferably the sprouts of these plants are used.

Comparatively large amount of sulforaphane are also contained in the sprouts of broccoli, radish, and purple cabbage.

Sulforaphane can be obtained from the above plants using a known extraction procedure. It may be purified or freeze-dried, if necessary.

The resultant extract should preferably contain sulforaphane of about 0.001-200 mg/g, preferably about 0.005-80 mg/g, and most preferably about 0.01-50 mg/g.

The intake of sulforaphane should be determined according to its application, age, gender, body weight, and state of health of the person who takes it, other conditions of the person, and severity of symptoms of the person. For example, the intake for an adult is preferably about 0.001-1,000 μg/kg/day, more preferably about 0.005-200 μg/kg/day, and most preferably about 0.01-60 μg/kg/day.

In addition to the above-mentioned sulforaphane, gelanylgelanyl acetone (GGA), which is a TRX inducer, may be used in various combinations.

The above extract may be used as it is or as pharmaceutical preparation containing TRX inducers as active ingredients. When the extract is used as pharmaceutical preparation, the preparation may be prepared with the vehicles mentioned in the section “TRX” above.

The agents containing TRX inducers as active ingredients according to the present invention may also be used as foods and beverages. When they are used as foods or beverages, they may be prepared by mixing the above extract with its source plants.

Such foods or beverages may, for example, be health foods, nutrition supplementing foods (nutrition balancing foods, supplements, etc.), nutritionally functional foods, specified health-promoting foods, and foods for the sick. The manufacturing processes of these foods are not particularly limited as long as its expected effect, i.e., induction of the thioredoxin expression, is obtained. These processes can be carried out using conventional methods.

Such foods and beverages include, for example, sweets such as chewing gum, candy, gummy, tablet candy, cookies, cake, chocolate, ice cream, jelly, mousse, pudding, biscuit, corn flakes, chewable tablets, wafers, and rice crackers; beverages such as carbonated beverages, soft drinks, milk-derived beverages, coffee-flavored beverages, black-tea flavored beverages, juice-containing beverages, nutritional beverages, alcoholic beverages, and mineral water; powdered beverages such as powdered juice and powdered soup; balancing nutritional foods; supplements in the forms of powders, capsules, and tablets; seasonings such as dressing and sauce; bread, noodles, pasted fish and meat such as kamaboko (i.e., steamed fish paste); and seasoned fish and vegetables for sprinkling over rice.

Preventive or therapeutic agents of the present invention may be prepared by combining polypeptide of the thioredoxin family (TRX) with thioredoxin inducers.

Some example of newly identified actions and effects of TRX will be described below. It should be understood, however, that the present invention is not limited to these examples. First, TRX suppresses the increase in neutrophils or in other lymphocytes that leads to the disorders caused by the toxic substances contained in smoke produced by the combustion of fibers like tobacco.

Increased neutrophils trigger inflammatory reaction and this reaction is one of the main factors of the disorders caused by toxic substances contained in smoke of fibers including tobacco. TRX suppresses such inflammatory reaction derived from the increased neutrophils by inhibiting the increase in neutrophils.

Since preventive or therapeutic agents of the present invention contain TRX and/or one or more of its inducers, they can control inflammation derived from the increase of neutrophils (not in particular organs but in various organs in the entire body) that is induced by toxic substances contained in smoke such as smoke of tobacco. Accordingly, the therapeutic or preventive agents of the present invention are effective against a wide range of disorders caused by such toxic substances.

Second, TRX prevents toxic substances contained in smoke produced by the combustion of fibers like tobacco from inducing the expression of TNF-α and MIF. TNF-α and MIF are early inflammatory cytokines.

Inflammatory reaction (systemic inflammatory reaction) caused by TNF-α and MIF is another factor of disorders induced by toxic substances contained in smoke such as smoke of tobacco. The expression of TNF-α and MIF is induced by such toxic substances. TRX suppresses the expression of these inflammatory cytokines (TNF-α and MIF) and consequently alleviates the inflammatory reaction in the body.

TNF-α plays an important role in systemic inflammatory reaction induced by smoking. The results of this study indicate that TRX prevents toxic substances contained in, for example, smoke of tobacco from inducing the expression of mRNA of TNF-α and the expression of TNF-α protein.

MIF, as well as TNF-α, plays an important role in systemic inflammatory reactions induced by smoking. MIF is known as a cytokine that is closely involved in delayed allergic reaction. MIF induces inflammation and immune reactions by gathering macrophages at the sites of inflammation. The results of this study indicated that TRX prevents toxic substances contained in smoke such as smoke of tobacco from inducing the expression of mRNA of MIF and the expression of MIF protein.

Since preventive or therapeutic agents of the present invention contain TRX and/or one or more of its inducers as active ingredients, they can suppress inflammatory reactions (such as inflammatory reactions caused by TNF-α or MIF) that are induced by toxic substances contained in, for example, smoke of tobacco (in the organs including lungs, bronchi, liver, heart, spleen, esophagus, and stomach). Therefore the preventive or therapeutic agents of the present invention are effective against a wide range of disorders caused by such toxic substances.

Third, TRX prevents toxic substances contained in smoke, produced by the combustion of fibers like tobacco, from inducing the expression of MMP-12.

Destruction of the pulmonary alveolar construction by MMP-12 in an early stage of inflammation and tissue fibrosis which is consequently induced by it are other factors of disorders induced by the toxic substances contained in, for example, smoke of tobacco. TRX prevents the toxic substances from inducing the expression of MMP-12 so as to alleviate tissue fibrosis and to prevent the destruction of the pulmonary alveolar construction in an early stage of inflammations

Tissue fibrosis is known as a common factor for all chronic diseases that occurs with infections, chronic diseases, and drug treatments. Tissue fibrosis is generally considered to be a late phase of inflammatory reaction of chronic inflammation.

MMP-12 is an enzyme that is primarily secreted by macrophages to decompose various extracellular matrices including elastin (present in large quantities in ligaments, lungs, and arteries). Since macrophages that secrete MMP-12 accumulate at the focuses of tissue inflammation, MMP-12 is closely related to the occurrence and progression of various inflammatory diseases (arthritis, pulmonary edema, aneurysm, atherosclerosis, etc.).

MMP-12 is known to cause disorders such as pulmonary edema, chronic bronchitis, and fibrosis by disturbing the balance between elastase and its inhibitors in the body if its activity is enhanced by smoking and the like, as well as to exacerbate chronic inflammatory diseases by promoting macrophage migration and infiltration (Transgenic Res 13:261-9, 2003).

Thus, inhibition of the MMP-12 activity contributes to the treatment of these diseases, all of which are caused by tissue fibrosis.

As mentioned above, MMP-12 plays an important role in reactions such as tissue fibrosis induced by toxic substances contained in smoke produced by the combustion of fibers like tobacco. The results of this study indicate that TRX suppresses the mRNA expression of MMP-12 induced by toxic substances contained in smoke such as smoke of tobacco.

Since the preventive or therapeutic agents of the present invention contain TRX and/or one or more of its inducers as active ingredients, they can prevent destruction of the pulmonary alveolar construction in an early phase of inflammation (such as inflammation caused by MMP-12), and they consequently suppress tissue fibrosis.

Therefore, the preventive or therapeutic agents of the present invention are effective against a wide range of disorders (particularly, chronic diseases) caused by toxic substances contained in smoke produced by the combustion of fibers. Additionally, it should be understood that TRX's suppression of the expression of MMP-12 means not only that TRX suppresses neutrophil infiltration but also that TRX has a preventive effect against destruction of the pulmonary alveolar construction and the progression of pulmonary edema.

The preventive or therapeutic agents of the present invention can be used for treating disorders induced by toxic substances contained in smoke produced by the combustion of fibers like tobacco. The term “disorders induced by toxic substances contained in smoke produced by the combustion of fibers”, refers to any disorders as long as they are characterized to be caused by inflammatory reactions and tissue fibrosis due to such toxic substances. Their examples will be given below.

Diseases

The preventive or therapeutic agents of the present invention can be used for treating disorders (e.g., inflammation) of the respiratory, cardiovascular, digestive organs, and brain that are caused by inhalation of toxic substances contained in smoke produced by the combustion of fibers. These disorders include those induced directly by intake of such toxic substances through respiratory organs, esophagus, stomach, as well as those induced indirectly in various organs and tissues due to such toxic substances after they are absorbed into the body. Such disorders also include generalized disorders (e.g., systemic inflammation, allergic diseases, and fetal anomalies) that are caused by toxic substances contained in smoke produced by the combustion of fibers.

The respiratory organs, as used herein, includes all organs related to alveolar gas exchange including nasal cavity, paranasal sinuses, pharynx, larynx, trachea, bronchi, lung, and the like. Respiratory diseases, as used herein, include pulmonary emphysema, chronic obstructive pulmonary disease (COPD) such as chronic bronchitis, bronchial asthma, bronchiestasis, chronic sinusitis, respiratory hypofunction, respiratory insufficiency, pneumonia (including eosinophilic pneumonia and interstitial pneumonia), pulmonary fibrosis, adult respiratory distress syndrome, chronic laryngitis, respiratory bronchiolitis, allergic rhinitis, and the like. Respiratory diseases also include oral disorders such as periodontitis and stomatitis.

The cardiovascular organs, as used herein, include all organs related to the circulation such as heart, blood vessels, and vascular endothelium. Examples of cardiovascular disorders are atherosclerosis, angina pectoris, myocardial infarction, and heart failure, but not limited to these.

The digestive organs, as used herein, include all organs related to digestion or absorption such as the esophagus, stomach, duodenum, small intestine, large intestine, liver, and the like. Examples of digestive organ diseases are gastric ulcer, duodenal ulcer, chronic gastritis, atrophic gastritis, erosive gastritis, colon polyps, hepatic fibrosis, and the like. Hepatic fibrosis is a phenomenon common among chronic liver diseases. The progression of hepatic fibrosis will increase the risk of liver cancer.

Brain diseases, as used herein, include cerebral infarction, subarachnoid hemorrhage, cerebrovascular dementia, and the like.

In addition, the preventive or therapeutic agents of the present invention can be used for treating malignant neoplasms such as lung cancer, oral cancer, pharyngeal cancer, cancer of the paranasal sinuses, esophageal cancer, stomach cancer, liver cancer, pancreatic cancer, renal pelvis cancer, and bladder cancer, and cancer of uterine cervix.

The preventive or therapeutic agents of the present invention are useful for managing disorders of the sensory organs such as macular degeneration, retinal infarction, retinal venous occlusion, cataract, glaucoma, reduced visual acuity, deafness, hyposmia, and amblygeustia.

Furthermore, the therapeutic agents of the present invention are effective for the treatment of mucus secretion such as sputum and peripheral vascular and circulatory disorders induced by temporary and massive inhalation or absorption of toxic substances contained in smoke produced by the combustion of fibers. In addition, the preventive or therapeutic agents of the present invention are effective against disorders induced by direct contact with smoke produced by the combustion of fibers. An example of such symptoms is airway burn.

TRX is known to be an early pregnancy factor and the serum concentration of TRX increases in an early stage of pregnancy. TRX is considered to be important for the maintenance of pregnancy because of its high expression in the placenta. Moreover, the low incidence of anomalies in mice excessively expressing TRX suggests the importance of TRX in the normal fetal development.

Therefore, the preventive or therapeutic agents of the present invention are effective for preventing or treating fetal disorders that occurs in a pregnant woman who has inhaled toxic substances contained in smoke. Examples of such fetal disorders include low birth weight, abortion, premature birth, perinatal death, congenital anomalies, delayed intrauterine development, pediatric cancer, infant sudden death syndrome, and the like.

The examples of the present inventions will be described in more detail below. It should be understood, however, that the present invention is not limited by these examples.

### EXAMPLE 1

In this example, C57BL/6 mice (used as controls; purchased from Shimizu Laboratory Supplies Co., Ltd.) and thioredoxin-transgenic mice (TRX-Tg; prepared at the Institute for Virus Research Kyoto University) (both 2-month-old males) were used.

Using an SIS-CS smoke exposure system (Shibata Scientific Technology, Ltd.), the entire bodies of the mice were exposed to smoke from 20 cigarettes diluted to 3% for 30 minutes twice at an interval of 30 minutes.

The mice were exposed to tobacco smoke under these conditions for three consecutive days.

Lung Tissue Disorders

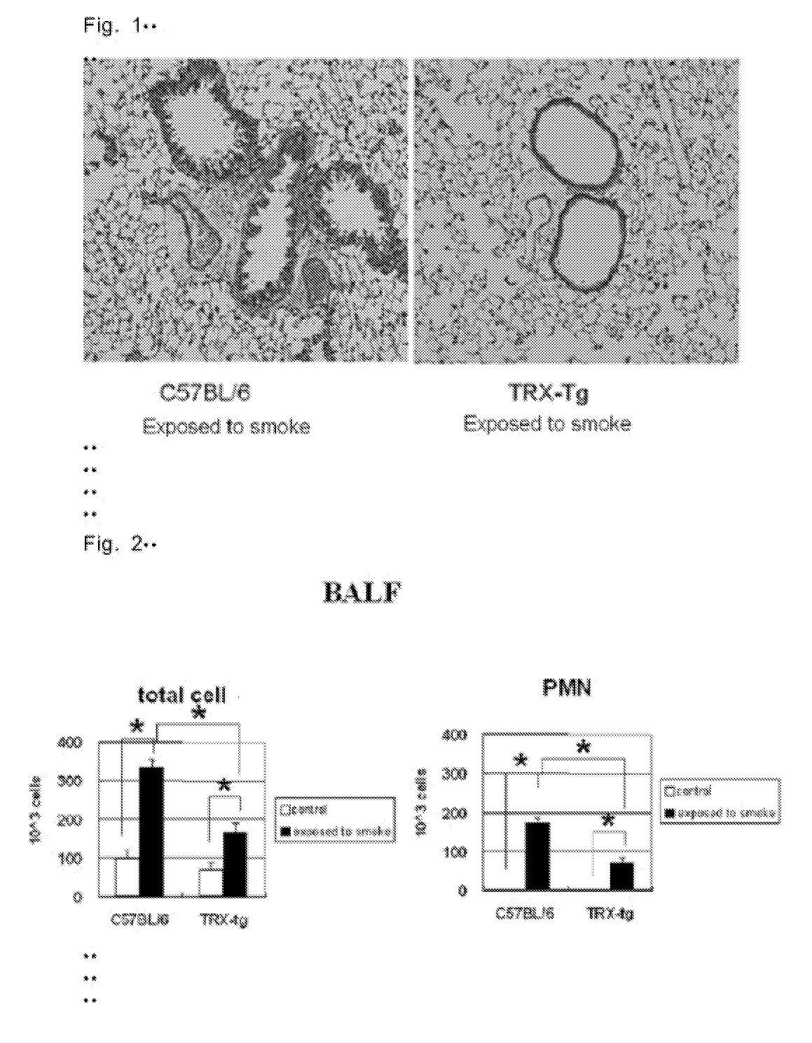

After exposed to tobacco smoke under the above-mentioned conditions, the left lungs of the mice were removed and fixed with formalin in an expanded state to prepare sections (4 μm). The sections were then stained with hematoxylin-eosin (HE). FIG. 1 shows the results.

In the bronchial epithelium of C57BL/6 mice, inflammatory cell infiltration suggested an enhancement of the mucoepithelial responsiveness. In contrast, in TRX-Tg mice, no symptoms that are characteristic of smoking-related disorders were noted even after exposure to tobacco smoke.

Counting of Inflammatory Cells in BALF

On the day after the above-described schedule of tobacco smoke exposure has completed, the mice were sacrificed to collect bronchoalveolar lavage fluid (BALF; 5 ml). Diff-Quik stained specimens were prepared using a Cytospin (Shandon) and then the total cell count and the neutrophil (PMN) count, which is an index of inflammation, were examined in the BALF. FIG. 2 shows the results.

The exposure to tobacco smoke increased the neutrophil count in the BALF. In the TRX-Tg mice, the increase in neutrophils in the BALF after smoking was significantly smaller than in the C57BL/6 mice.

The results of examination of lung tissue disorders and counting of inflammatory cells in BALF suggested that TRX is effective for preventing toxic substances in the tobacco smoke, from inducing inflammation in the organs directly stimulated by the smoke. These organs include lungs, bronchi, esophagus, stomach, and the like.

### EXAMPLE 2

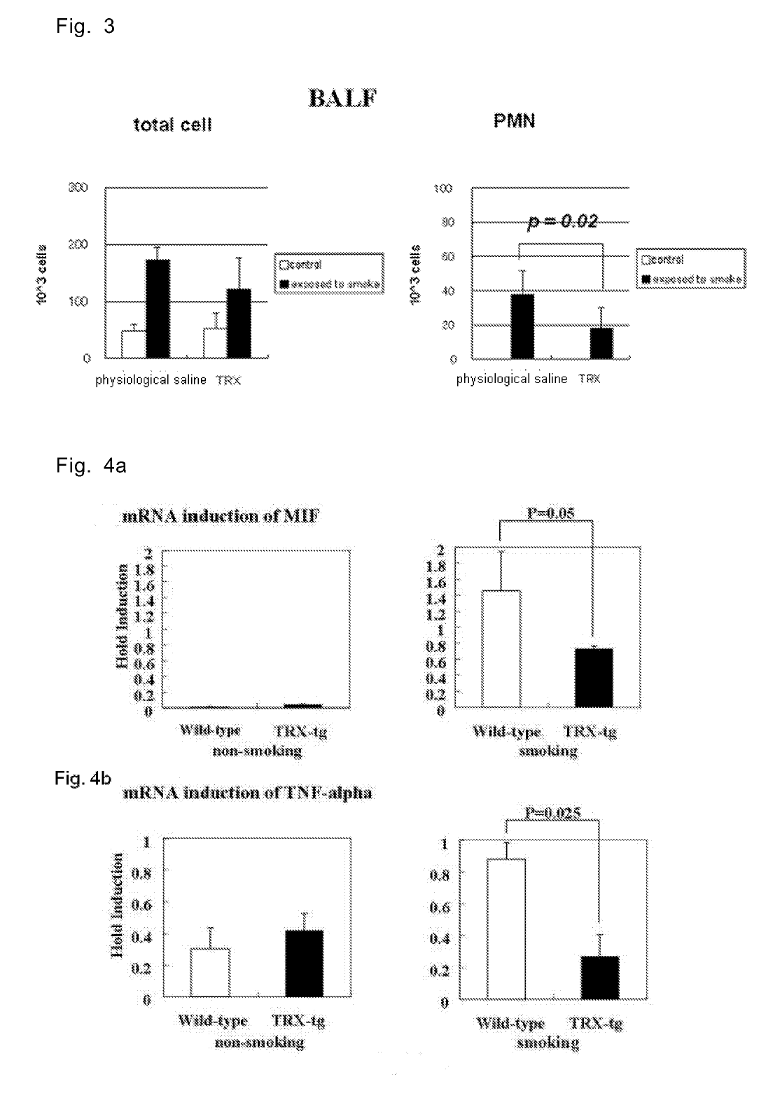

TRX was administered to wild-type mice to examine whether the administered TRX inhibits the increase in neutrophils in BALF due to exposure to tobacco smoke. To C57BL/6 mice (7-week-old males), TRX was administered at 20 μg/0.1 ml physiologic saline immediately before and 3 hours after exposure to tobacco smoke (TRX-treated group: total dose 40 μg/day/mouse). C57BL/6 mice that were administered physiologic saline by the same schedule as that for TRX-administered mice were used as a control group. The animals were exposed to tobacco smoke similarly to Example 1. On the day after the smoke exposure schedule completed, the mice were sacrificed to collect BALF (5 ml). FIG. 3 shows the results.

As shown in FIG. 3, the total cell count in BALF tended to be smaller in the group that was treated with TRX after smoke exposure (TRX/smoke exposure group in FIG. 3) than in the group that was treated with saline after smoke exposure (saline/smoke exposure in FIG. 3). The PMN count was significantly lower in the TRX/smoke exposure group than in the control group.

Therefore, TRX was suggested to be effective for preventing the toxic substances in tobacco smoke from causing inflammation in the organs directly stimulated by the smoke. These organs include lungs, bronchi, esophagus, stomach, and the like.

### EXAMPLE 3

C57BL/6 mice (used as controls; purchased from Shimizu Laboratory Supplies Co., Ltd.) and thioredoxin-transgenic mice (TRX-Tg; prepared at the Institute for Virus Research Kyoto University) (both males aged 9-12 weeks) were used. Using an SIS-CS tobacco smoke exposure system (Shibata Scientific Technology, Ltd.), the whole bodies of the mice were exposed to smoke from 20 cigarettes diluted to 3% for 30 minutes twice at a 30-minute interval. The animals were exposed to tobacco smoke under these conditions on three consecutive days.

Extraction of Total RNA and Real-Time RT-PCR Analysis

Total RNA was extracted from the spleens of the mice exposed to tobacco smoke as above using Rneay Mini Kits (Qiagen). The procedure of total RNA extraction was performed according to the protocol described in the package insert of Rneay Mini Kit. 1 μg of the obtained RNA was used for the synthesis of single chain cDNA. Reverse transcription reaction was performed in a 20-μL reaction mixture consisting of 1 μg RNA, 250 ng random hexamer, 40 units of Rnase OUT inhibitor, and Super Script III Rnase H minus reverse transcriptase (Invitrogen).

After reverse transcription reaction, all cDNA obtained was used for real-time quantitative PCR, in which the expression of mouse MIF gene and of mouse TNF-α gene was detected by TaqMan reverse transcription PCR (RT-PCR) using an ABI Prism 7000 sequence detection system (Applied Biosystem). FIG. 4 shows the results.

As shown in FIG. 4a, the transcription activity of mRNA of MIF, which is a (early) inflammatory cytokine, was more significantly suppressed (p=0.05) in the smoke-exposed TRX-Tg group than in the smoke-exposed control group (wild-type).

As shown in FIG. 4b, the transcription activity of mRNA of TNF-α, which is a (early) inflammatory cytokine, was more significantly suppressed (p=0.025) in the smoke-exposed TRX-Tg group than in the smoke-exposed control group (wild-type).

These results suggest that TRX prevents toxic substances in smoke, produced by the combustion of fibers like tobacco, from inducing the expression of mRNA of inflammatory cytokines (MIF, TNF-α).

### EXAMPLE 4

Western Blotting Analysis

Similarly to the above real-time RT-PCR analysis, C57BL/6 mice (wild-type group, n=3) and thioredoxin-transgenic mice (TRX-Tg group, n=3) (both males aged 9-12 weeks) were used in this example. The whole bodies of the animals were exposed to smoke from 20 cigarettes diluted to 3% for 30 minutes twice at a 30-minute interval. The animals were exposed to tobacco smoke under these conditions on three consecutive days. Then, the spleen was removed from the animals of each group and used for the analyses described below.

Western blotting was performed using anti-mouse MIF polyclonal antibody and anti-mouse MIF polyclonal antibody (both from Cosmo Bio Co., Ltd.) according to the method of Towbin et al. (1979).

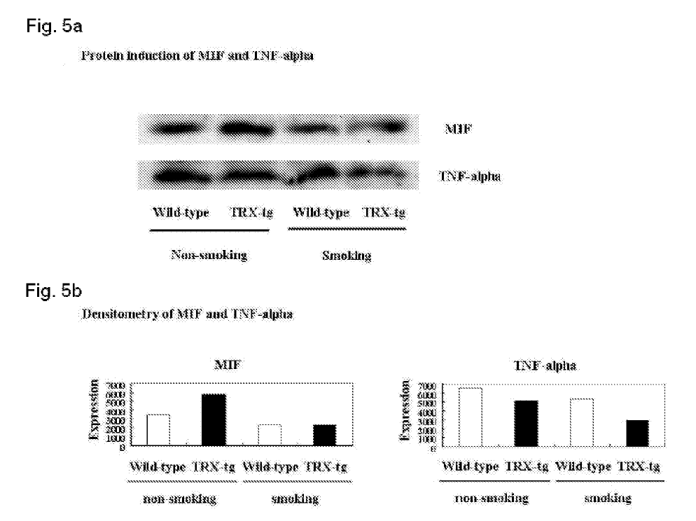

FIG. 5a shows the results, which confirmed the expression of mouse MIF protein and TNF-α protein.

FIG. 5a shows the expression of MIF protein and TNF-α protein in spleen tissues of smoke-exposed TRX-Tg mice (n=3) and control C57BL/6 mice (wild-type, n=3) observed by Western blotting.

As shown in the figure, the MIF protein expression level after exposure to smoke was much more reduced in the TRX-Tg mice than in C57BL/6 mice.

Similarly, the TNF-α protein expression level was more reduced in the TRX-Tg mice than in the C57BL/6 mice.

FIG. 5b shows the expression-activities of MIF protein and TNF-α protein in spleen tissues of smoke-exposed TRX-Tg mice and of control C57BL/6 mice. The expression-activities were examined by densitometry.

As shown in the figure, the MIF protein and TNF-α protein expression levels were more reduced in the smoke-exposed TRX-Tg mice than in the control C57BL/6 mice.

These results suggest that TRX prevents toxic substances in smoke, produced by the combustion of fiers like tobacco, from inducing the expression of MIF protein and TNF-α protein, both inflammatory cytokines.

### EXAMPLE 5

OxyBlot Analysis

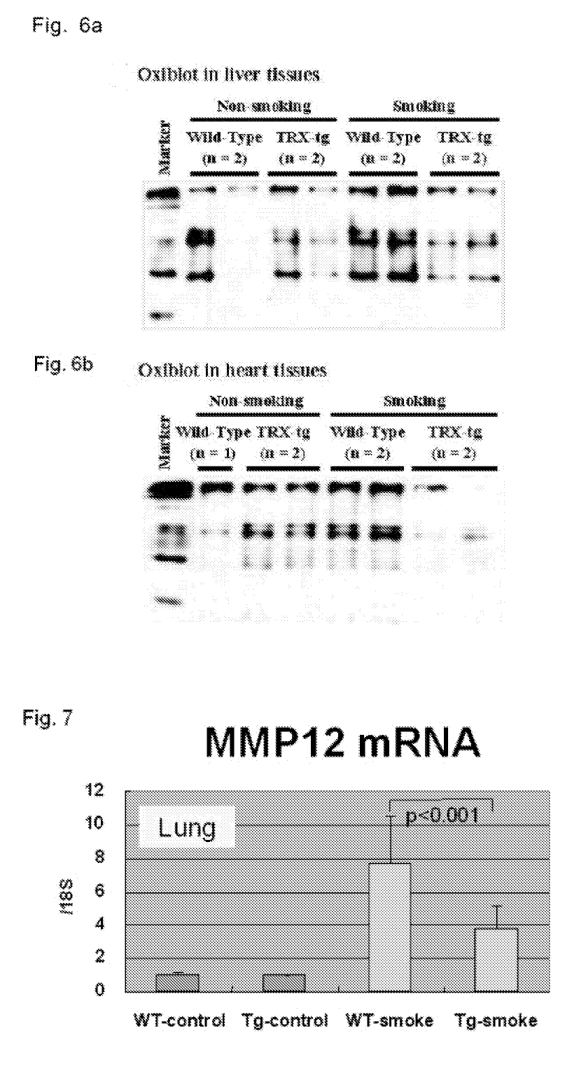

OxyBlot analysis was performed in the heart and liver of smoke-exposed TRX-Tg mice (n=3) and C57BL/6 mice (n=3, control group) to evaluate oxidative stress in organs other than the spleen (which was evaluated by Western blotting). In OxyBlot analysis, the carbonyl radical in oxidized protein molecules was allowed to react with 2,4-dinitrophenylhydrazine (DNPH), and the resultant 2,4-dinitrophenylhydrazone (DNP-hydrazone) is detected by chemiluminescence using DNP-specific antibody and secondary antibody (HRP-labeled antibody). OxyBlot analysis was performed using OxyBlot Protein Oxidation Detection Kit (Invitrogen), which is a kit for the analysis of the oxidation level of protein by Western blotting. The exampleal procedure was performed according to the protocol described in the package insert of the kit. FIG. 6 shows the results.

FIG. 6 shows oxidative stress evaluated in the heart (FIG. 6a) and liver (FIG. 6b) of the smoke-exposed TRX-Tg mice (n=3) and the control C57BL/6 mice (n=3). The oxidation level of protein was increased in the heart (FIG. 6a) and liver (FIG. 6b) by smoking, but this increase was smaller in the TRX-Tg mice than in the control mice.

These results suggest that TRX suppresses oxidative stress induced by toxic substances in smoke, produced by the combustion of fibers like tobacco.

### EXAMPLE 6

MMP-12 Suppression Experiment

In this example, the whole bodies of both C57BL/6 mice (control group, n=3) and thioredoxin-transgenic mice (TRX-Tg group, n=3) (both males aged 9-12 weeks) were exposed to smoke from 20 cigarettes diluted to 3% for 30 minutes twice at a 30-minute interval on three consecutive days before use.

The lungs were removed from each group and used for the analysis described below.

MMP-12 suppression was evaluated by real-time RT-PCR analysis.

Total RNA was extracted from the lungs of mice exposed to tobacco smoke as above using a Rneay Mini Kit (Qiagen) according to the protocol described in the package insert of the kit. 1 μg of the obtained RNA was used for the synthesis of single chain cDNA, and, after reverse transcription reaction, real-time quantitative PCR was performed. In the real-time quantitative PCR, the expression of mouse MMP-12 gene was detected using ABI Prism 7000 sequence detection system (Applied Biosystems) by Taq Man reverse transcription PCR (RT-PCR).

FIG. 7 shows the results.

The transcription activity of mRNA of MMP-12, which is a fibrosis-promoting factor, was more significantly suppressed in the smoke-exposed TRX-Tg group (n=3) than in the smoke-exposed control group (n=3).

These results suggest that TRX suppresses inflammation induced by toxic substances in smoke, produced by the combustion of fibers like tobacco and that TRX suppresses fibrosis associated with the inflammation.

### EXAMPLE 7

C57BL/6 mice (used as controls; purchased from Shimizu Laboratory Supplies Co., Ltd.) and thioredoxin-transgenic mice (TRX-Tg; prepared at the Institute for Virus Research Kyoto University) were used as experimental animals. Two-month-old males were acclimated in an ordinary animal room for 1 month, and then the turned-3-month-old mice were exposed to tobacco smoke.

They were exposed to smoke from 10 cigarettes for 50 minutes per day using an SIS-CS smoke exposure system (Shibata Scientific Technology, Ltd.).

The above smoke-exposure protocol was performed to adjust the blood HbCO to 10% of the smoker level.

Mouse Smoking-Induced Pulmonary Emphysema Model

The left lungs of the mice exposed to tobacco smoke as above were fixed in an expanded state with 25-cm H₂O OCT, and frozen sections (5 μm) were prepared.

These sections were stained with hematoxylin and morphologically evaluated.

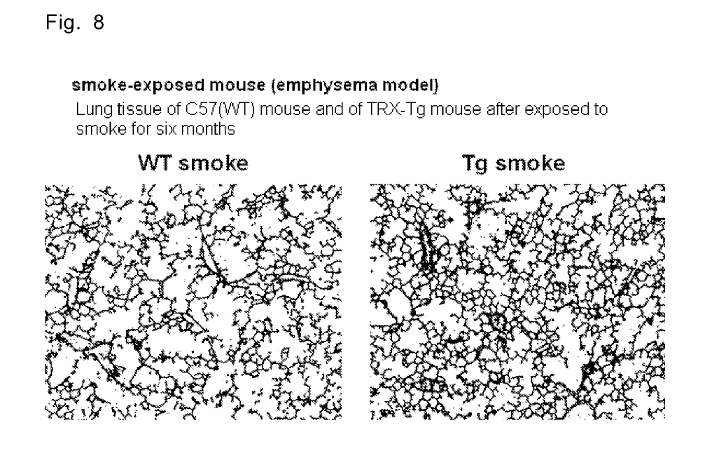

In the C57BL/6 mice (wild-type), marked enlargement of air spaces and interruption of the alveolar wall, both characteristics of pulmonary emphysema, were observed after 6 month-exposure to tobacco smoke. FIG. 8 shows the results.

Comparing the lung tissues of C57BL/6 (wild-type) and TRX-Tg mice after 6 month-exposure to tobacco smoke (FIG. 8), marked enlargement of air spaces and interruption of the alveolar wall were noted in the C57BL/6 mice while they were mild in the TRX-Tg mice.

Therefore, in a pulmonary emphysema model mice prepared by 6-month exposure to tobacco smoke, which reflects a more chronic pathological state (than the model prepared by 3-day exposure to tobacco smoke), pulmonary emphysema was mitigated in TRX-Tg mice.

These results established the therapeutic effect of TRX against disorders (particularly chronic disorders) due to smoking.

Since pulmonary emphysema is one of the major pathologic features of chronic obstructive pulmonary disease (COPD), the results of Example 7 directly evidence the effectiveness of TRX as a preventive or therapeutic agent against COPD.

On the basis of the results of Examples 1-7 above, it is concluded that TRX is effective for the control of a wide range of disorders (acute and chronic diseases) induced by toxic substances contained in smoke produced by the combustion of fibers like tobacco.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

1. A therapeutic agent for disorders caused by toxic substances contained in smoke produced by combustion of fibers, comprising an effective amount of at least one material selected from a group consisting of polypeptides of the thioredoxin family and inducers thereof, as active ingredients, wherein said inducer is at least one selected from sulforaphane or gelanylgelanyl acetone.

2. The therapeutic agent of claim 1, wherein said polypeptide of the thioredoxin family is human thioredoxin.

3. The therapeutic agent of claim 1, wherein said fiber is selected from a group consisting of tobacco, wood, and paper.

4. The therapeutic agent of claim 1, wherein said disorders are inflammation in at least one organ selected from a group consisting of respiratory organs and digestive organs.

5. The therapeutic agent of claim 4, wherein said respiratory organs are at least one organ selected from a group consisting of nasal cavity, paranasal sinuses, pharynx, larynx, trachea, bronchi, and pulmonary alveoli.

6. The therapeutic agent of claim 4, wherein said digestive organs are at least one organ selected from a group consisting of esophagus, stomach, duodenum, liver, small intestine, and large intestine.

7. The therapeutic agent of claim 1, wherein said disorder is chronic obstructive pulmonary disease or bronchial asthma.

8. A therapeutic formulation for disorders caused by toxic substances contained in smoke produced by combustion of fibers, comprising an effective amount of at least one material selected from a group consisting of polypeptides of the thioredoxin family and inducers thereof, as active ingredients, wherein said inducer is at least one selected from sulforaphane or gelanylgelanyl acetone.

9. The formulation of claim 8, wherein said inducer is sulforaphane.

10. The formulation of claim 8, wherein said therapeutic formulation is foods or beverages.
